(19) United States
(12) Patent Application Publication
(10) Pub. No.: US 2015/0065943 A1
(43) Pub. Date: Mar. 5, 2015

(54) Method to Accelerate Healing

(71) Applicant: Matthew DeBow, Buda, TX (US)

(72) Inventor: Matthew DeBow, Buda, TX (US)

(73) Assignee: Matthew DeBow, Buda, TX (US)

(21) Appl. No.: 14/011,458

(22) Filed: Aug. 27, 2013

(51) Int. Cl. A61N 5/06; A61K 33/00

(52) U.S. Cl. CPC A61N 5/0622; A61K 33/00
USPC 604/20

(57) ABSTRACT

The invention combines light and compounds to accelerate healing. This photobiolocical treatment system activates cells at a molecular level after the ingestion or application of nitric oxide activators then shining light on afflicted area, resulting in faster recovery. Isolated wavelengths of monochromatic biostimulatory light and vasodilating compounds are independently are known to increase and accelerated cellular metabolism and promote faster than normal recovery. Utilizing both simultaneously activates a synergistic biological effect on cells at a molecular level speeding the injured biological living tissue recovery time of patients.

### FIELD OF THE INVENTION

The invention relates generally to methods and compositions for the improvement and acceleration of healing. More particularly, the invention relates to compositions and methods that employ certain types of light and nitric oxide activators in combination to accelerate the healing process.

### BACKGROUND OF THE INVENTION

Nitric oxide (NO) affects the wound healing process by its wide-ranging impact on cellular activities, including antimicrobial and vasodilatory effects, modulation of platelet/cytokine function, and promoting angiogenesis and matrix deposition (Isenberg et al. 2005).

Light therapy, also known as phototherapy, consists of exposure to daylight or to specific wavelengths of light for photobiological treatment of various ailments including skin disorders, pain management and wound healing. Case studies have found low-level laser light to be helpful as an adjunctive treatment in wound healing (Sutterfield 2008).

### SUMMARY OF THE INVENTION

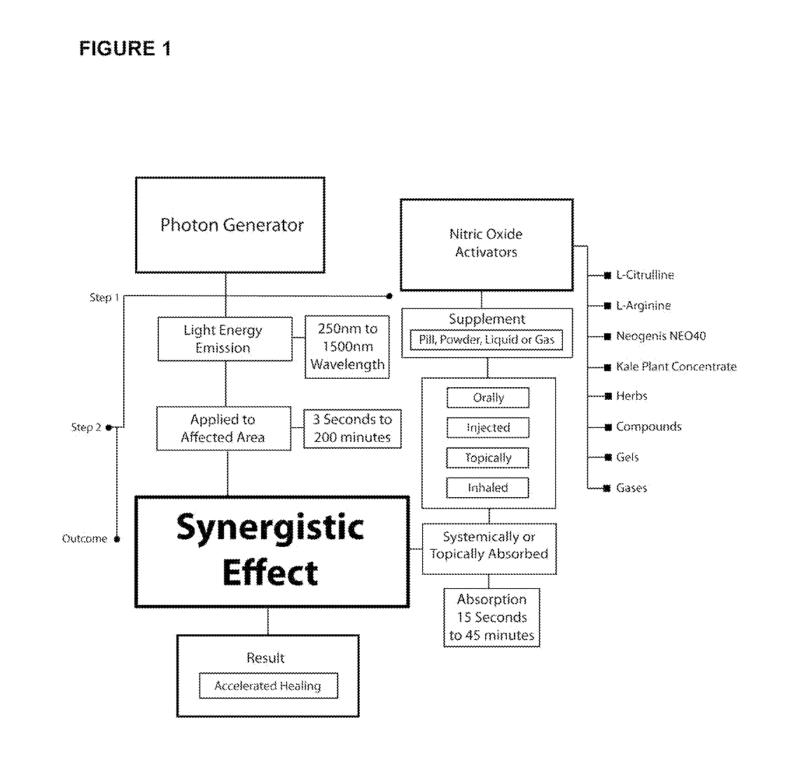

FIG. 1 Process Diagram—For the specific uses of nitric oxide activators in combination with both visible and non-visible light.

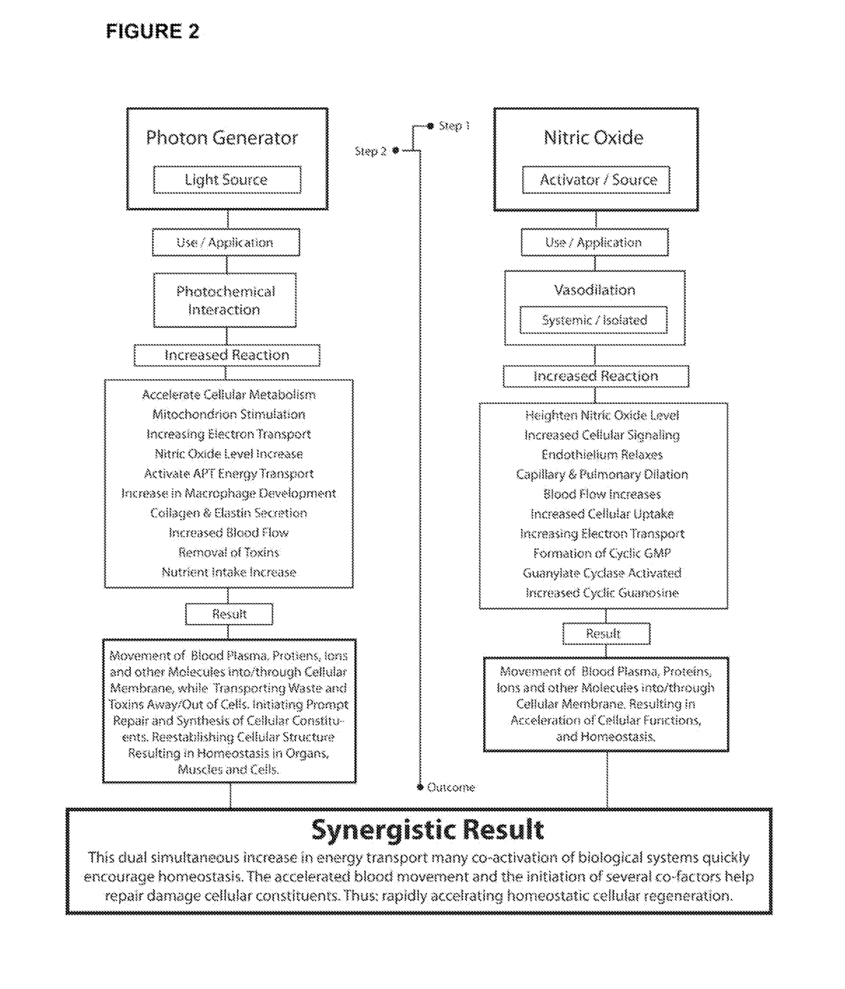

FIG. 2. Synergistic Mechanism—How specific waves of light and nitric oxide work together at a cellular, molecular and energy level.

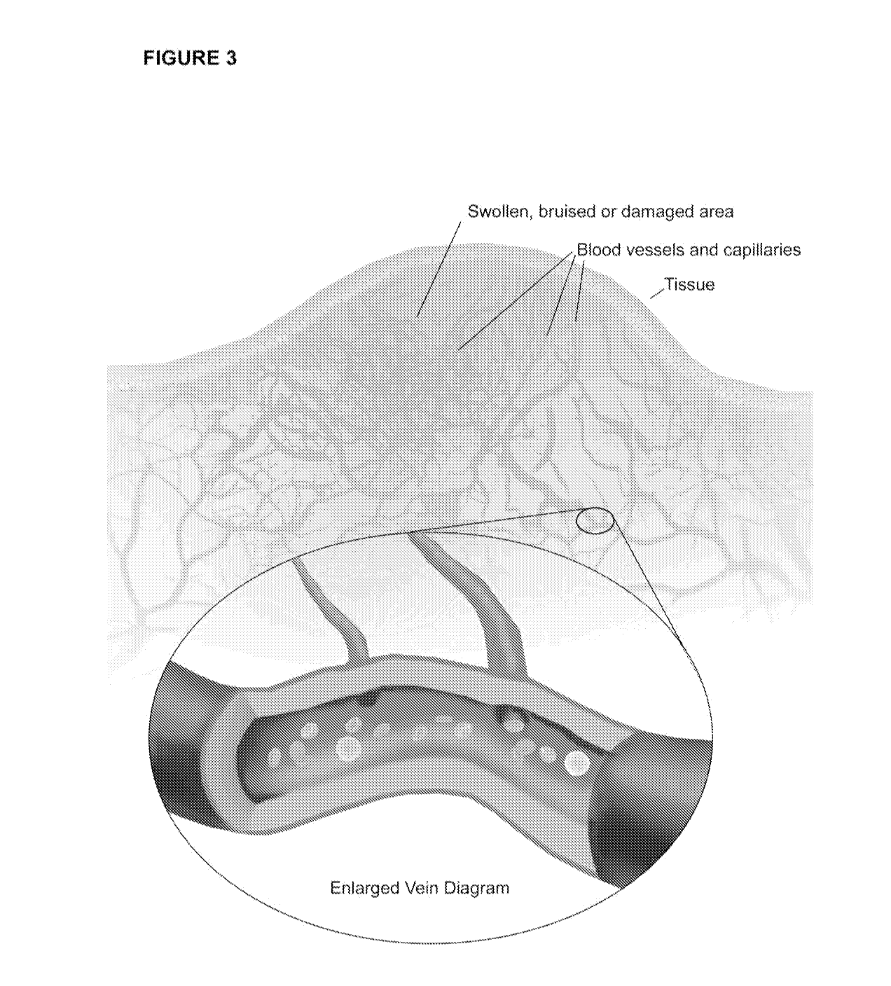

FIG. 3. Illustration of swollen, bruised or damaged untreated area.

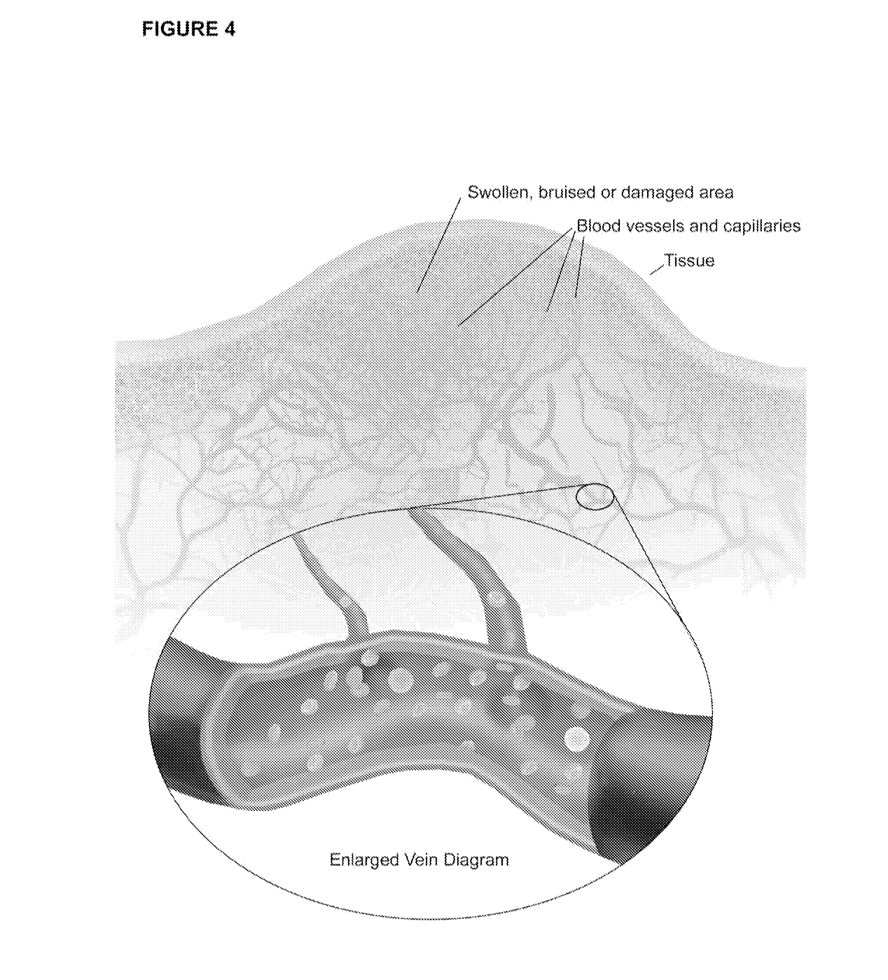

FIG. 4. Illustration of how Nitric Oxide Supplements Increases Blood Flow (vasodilatation of veins and capillaries). With nitric oxide supplementation blood vessels dilate, blood flow increases, getting more nutrients to damaged cells, removing toxins, resulting in accelerated healing.

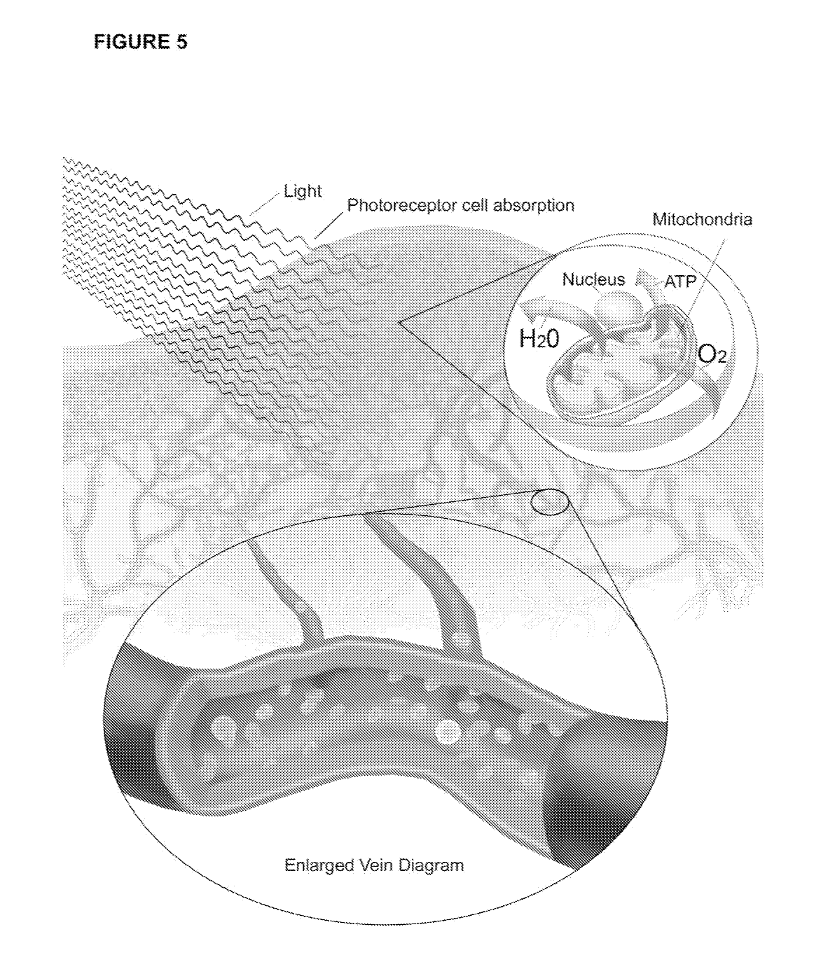

FIG. 5. Illustration of how Light Therapy Enhances Function (accelerating cell healing process). Light irradiation enhances cellular metabolism. Light triggers the conversion and release of APT and H20, and activates mitochondrial activity. Toxins are carried away with H20, oxygen is increased, growth factor production and cell proliferation is enhanced and healing is increased at a cellular level. Light Therapy Enhances Function (accelerating cell healing process).

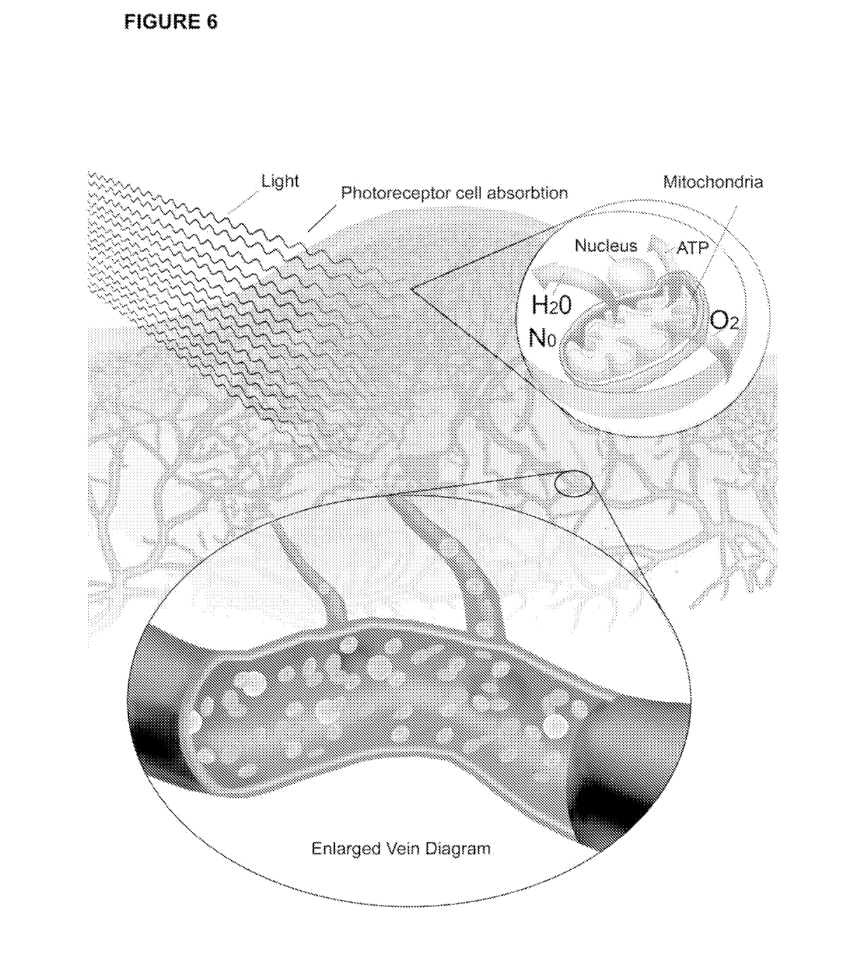

FIG. 6. Illustration of Maximum Accelerated Healing effect (combined light therapy & nitric oxide supplementation). Light irradiation enhances cellular metabolism. Light triggers the conversion and release of APT and H20, and activates mitochondrial activity. Toxins are carried away with H20, oxygen is increased, growth factor production and cell proliferation is enhanced and healing is increased at a cellular level. With nitric oxide supplementation blood vessels dilate, blood flow increases, getting more nutrients to damaged cells, removing toxins, resulting in accelerated healing.

The invention relates to compositions and methods that employ certain types of light and nitric oxide activators in synergistic combination to accelerate the healing process. Provided herein is a method to accelerate healing by administering an effective amount of a nitric oxide activator in combination with an effective amount of light therapy, so that healing is accelerated in a patient.

### DETAILED DESCRIPTION OF THE INVENTION

It has been discovered that the use of light therapy in combination with administration of oral supplements that increase levels of nitric oxide or activate nitric oxide in the body improves wound healing when compared to either therapy alone. In some embodiments, the combination of light therapy and nitric oxide activation or supplementation provides additive effects on wound healing. In other embodiments, the combination of light therapy and nitric oxide activation or supplementation provides synergistic effects on wound healing.

While not wishing to be bound by theory, it is believed that light therapy photobiologically causes increased cellular metabolism and nitric oxide causes vasodilation. Specific wavelengths of isolated and/or monochromatic light activate cellular metabolism catalyzing the movement of blood and nutrients into cells, thus providing new blood and removing toxins at a faster rate than would otherwise occur. When this photobiological therapy is done in the context of high levels of nitric oxide, the functionally of the light is increased, in turn hyper-accelerating the healing response.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

The articles “a” and “an” are used herein to refer to one or to more than one (i.e. to at least one) of the grammatical object of the article. By way of example, “a supplement” means one supplement or more than one supplement.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “comprises”, “comprising”, and the like can have the meaning ascribed to them in U.S. Patent Law and can mean “includes”, “including” and the like. As used herein, “including” or “includes” or the like means including, without limitation.

An “effective amount” generally means an amount that provides the desired local or systemic effect and/or performance. For example, an “effective amount” of a nitric oxide activator or an “effective amount” of light application, is an amount effective to accelerate or improve the healing of a wound.

As used herein, the term “healing” is the process of the restoration of tissue to its natural state from a wounded state. Healing involves the repair of living tissue, organs or the biological system as a whole and resumption of normal functioning. During healing, cells in the body regenerate and repair to reduce the size of a damaged or necrotic wound area and replace it with new living tissue. The replacement can happen in two ways: by regeneration in which necrotic cells are replaced by new cells that form similar tissue as was originally there; or by repair in which injured tissue is replaced with scar tissue. “Healing” may be indicated by a decrease in wound size, numbers of necrotic cells compared to numbers of new or scar tissue cells, or a change in a patient's perception of pain or other symptom associated with a wound.

As used herein, the term “light therapy,” also known as “phototherapy,” consists of exposure to daylight or to specific wavelengths of light to result in one or more photobiological effects. Light therapy may be carried out with the use of lasers, light-emitting diodes, fluorescent lamps, dichroic lamps or exposure to sunlight.

As used herein, “photobiology,” “photobiological response” and “photobiological treatment” refer to biological phenomena resulting from exposure to light. Photobiological responses are the result of chemical and/or physical changes induced in biological systems by exposure to light. In some embodiments, a photobiological response is an improvement, such as acceleration, in wound healing, and photobiological treatment is the treatment carried out to cause such a response.

A “subject” or “patient” is a vertebrate, including a mammal, such as a human. Mammals include, but are not limited to, humans, farm animals, sport animals and pets.

As used herein, the terms “supplement,” “drug,” “agent,” “compound,” and “activator” encompass any composition of matter or mixture which provides some pharmacologic effect that can be demonstrated in vivo or in vitro. This includes small molecules, nucleic acids, antibodies, microbiologicals, vaccines, vitamins, and other beneficial agents. As used herein, the terms further include any physiologically or pharmacologically active substance that produces a localized or systemic effect in a patient. In some embodiments, the pharmacologic effect is production of nitric oxide. In some embodiments, the pharmacologic effect is activation of nitric oxide. In other embodiments, the pharmacologic effect is increased metabolism of nitric oxide.

As used herein, “treat,” “treating” or “treatment” includes treating, ameliorating, or inhibiting a disease related condition and/or a symptom of a disease related condition. In some embodiments, “treating” a wound means improving or accelerating the time a wound requires to heal, or improving the symptoms associated with a wound.

As used herein, the term “wound” means an injury in which bodily tissue is torn, cut or punctured (an open wound), or where blunt force trauma causes a contusion (a closed wound). Open wounds include, but are not limited to, incisions, cuts, splinters, lacerations, tears, abrasions (grazes), punctures, and gunshot wounds. Closed wounds include, but are not limited to, contusions (bruises), hematomas and crush injuries. In some cases, a wound is an injury which damages the dermis of the skin. In some cases, a wound is an area of the body or a group of cells in the body that are not operating homeostatically.

### Light

Exposure to light, also known as light application, is employed in the methods of the instant invention. Light is contemplated to be applied to any part of the body of the patient, including the head, face, hands, arms, legs, back, chest, stomach, feet, buttocks and genitals to cause a photobiological response. In some embodiments, light is applied to the site of a wound to cause a photobiological response. In other embodiments, light is applied to a site separate from a wound site to cause a photobiological response.

Various wavelengths of light are contemplated for use in the instant invention, including ultraviolet light, visible light and infrared light. Visible light has a wavelength in the range of about 380 nanometers (nm) to about 740 nanometers. In some embodiments, the wavelengths of light used to hyperaccelerate the healing response are from 630 nanometer to 990 nanometer. This includes a range of 360 nanometers or 3600 angstroms. In some embodiments, the wavelengths of light employed in the instant invention are between 250 nm and 1500 nm. Sources of specific wavelengths of light are well known to those of skill in the art. Depending on the wavelength of light desired, various apparatus including but not limited to: photon generators, filters, lasers, light-emitting diodes, fluorescent lamps, dichroic lamps and tanning beds are employed. Exposure to sunlight is also contemplated for use in the methods described herein. Exposure to intense pulsed light is also contemplated for use in the methods described herein. In some embodiments, pulse rates are between 1 Hz and 1,000,000 Hz. Light isolated with or filtered through a prism is also contemplated for use in the instant invention. All types of light as described herein can cause photobiological effects in a patient.

Without wishing to be bound by theory, it is believed that exposure to a light source causes one or more of the following results: accelerating cellular metabolism, stimulating mitochondria, increasing electron transport, increasing nitric oxide levels, activating ATP energy transport, increasing macrophage development, secreting collagen and elastin, increasing blood flow, removing toxins, and increasing nutrient intake.

The appropriate number of light applications is contemplated to be determined relative to the severity of the pathology and may vary in frequency and length of application. In some embodiments, light is topically applied to a patient for approximately 3 seconds to approximately 200 minutes.

### Nitric Oxide

Nitric oxide, also known as nitrogen monoxide, is a free radical molecule with formula NO. In mammals, nitric oxide is an important cellular signaling molecule involved in many physiological and pathological processes. Increased levels of nitric oxide are contemplated for use in combination with light therapy as described herein in the treatment of wounds. Specifically, nitric oxide and light therapy are employed together to accelerate wound healing. Essentially any substance that increases levels of nitric oxide in the body is contemplated for use in the methods described herein, including but not limited to oral supplements. Nitric oxide is also contemplated to be increased in the body by injection, use of a topical solution and by inhalation.

In some embodiments, nitric oxide is increased in the body by orally ingesting particular supplements. The oral consumption of L-Arginine, L-Citrulline, beet extract, Neogenis NEO-40, kale concentrate, and other herbs in supplemental form that increase levels of nitric oxide are all contemplated for use in the methods described herein, separately and in combination.

Without wishing to be bound by theory, it is believed that increasing nitric oxide levels in a patient causes one or more of the following results: increasing cellular signaling, relaxing endothelium, dilating capillaries and pulmonary tissue, increasing blood flow, increasing cellular uptake, increasing electron transport, forming cyclic GMP, activating guanylate cyclase, and increasing cyclic guanosine levels.

### Dosage/Administration

Methods of delivery of a supplement or compound for activating or increasing nitric oxide in order to accelerate or improve the healing process according to the invention include but are not limited to, oral, topical, intra-arterial, intramuscular, subcutaneous, intravenous, intranasal, and inhalation routes. In certain embodiments, the delivery route is oral. Suitable modes of delivery will be apparent based upon the particular supplement or compound or combination thereof and their known administration forms. A nitric oxide activator or supplement may be administered by any suitable route, including without limitation: oral, rectal, nasal, topical (including transdermal, aerosol, buccal and sublingual), vaginal, penile, parenteral (including subcutaneous, intramuscular, intravenous and intradermal) and pulmonary.

For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, lozenges, powders and the like. Such compositions and preparations typically contain at least 0.1% of active compound. The percentage of the compositions and preparations can vary and may conveniently be from about 2% to about 60% of the weight of a given unit dosage form. The amount of active compound in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The nitric oxide supplement or nitric oxide activator may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

For topical administration, compounds may be applied in pure form, e.g., when they are liquids. However, it will generally be desirable to administer the active agent to the skin as a composition or formulation, for example, in combination with a dermatologically acceptable carrier, which may be a solid or a liquid.

Useful solid carriers include finely divided solids such as talc, clay, microcrystalline cellulose, silica, alumina, and the like. Useful liquid carriers include water, dimethyl sulfoxide (DMSO), alcohols, glycols, or wateralcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see Jacquet et al. (U.S. Pat. No. 4,608,392), Geria (U.S. Pat. No. 4,992,478), Smith et al. (U.S. Pat. No. 4,559,157), and Wortzman (U.S. Pat. No. 4,820,508). Such dermatological compositions can be used in combinations with the compounds described herein.

Therapeutic amounts can be empirically determined and may vary with the particular condition being treated, the subject, the particular formulation components, dosage form, and the like. The actual dose to be administered may vary depending upon the age, weight, and general condition of the subject as well as the severity of the condition being treated, along with the judgment of the health care professional. Therapeutically effective amounts can be determined by those ordinarily skilled in the art, and will be adjusted to the requirements of each particular case.

In embodiments employing oral supplements, the supplements are ingested less than an hour before the application of light. In some embodiments, the supplements are orally ingested 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes before the application of light. In preferred embodiments, the supplements are ingested 25 to 40 minutes before the application of light. In other embodiments, the supplements are ingested at a time that allows optimal levels of nitric oxide to be present in the body at the same time as the patient is exposed to the light.

In certain embodiments, the amount of supplement to be ingested is from 750 mg to 10,000 mg, varying due to the person's weight and severity of the pathology. In embodiments where the supplement is applied topically or inhaled, light is applied to the patient immediately after application or inhalation. The treatment application of red, infrared or blue light is 3 seconds to 55 minutes. Red light falls in the range of 400-484 THz frequency and 620-750 nm wavelength; blue light falls in the range of 606-668 THz frequency and 450-495 nm wavelength. Infrared light falls in the range of 405 THz-300 GHz frequency and 750 nm-1 mm wavelength. In preferred embodiments, light is applied directly to skin pathology. The amount of power is from 5 mW to 100 W.

In some embodiments, light is applied once per day. In other embodiments, light is applied twice per day. In preferred embodiments, light is applied three times per day. The appropriate number of light applications is contemplated to be determined relative to the severity of the pathology and may vary in frequency and length of application. In some embodiments, light is applied to a patient for approximately 3 seconds to approximately 200 minutes.

The combination of nitric oxide activators with the application of light is contemplated to be carried out in various time frames, at various time frequencies, at varying pulse rates and in varying durations, depending on the severity and nature of the condition being treated.

### BIBLIOGRAPHY

Isenberg J S et al. Nitric oxide in wound-healing. Microsurgery 2005; 25(5):442-51.

Sutterfield R. Light therapy and advanced wound care for a neuropathic plantar ulcer on a Charcot foot. J Wound Ostomy Continence Nurs 2008:35 (1): 113-115.

What is claimed is:

1. A method to accelerate healing comprising administering to a subject in need thereof an effective amount of a nitric oxide activator in combination with an effective amount of light therapy, so that healing is accelerated.
